(19) United States
(12) Patent Application Publication
(10) Pub. No.: US 2014/0371280 A1
(43) Pub. Date: Dec. 18, 2014

(54) Method of treating diabetic peripheral neuropathy through amendment of pre-neuronal diabetic coagulative micro-angiopathy and neuronal deficiency of mitochondrial ATP production, and through induction of neuronal regeneration

(71) Applicants: Shinichi Okuyama, Sendai (JP); Jessi Lu, Angleton, TX (US)

(72) Inventors: Shinichi Okuyama, Sendai (JP); Jessi Lu, Angleton, TX (US)

(21) Appl. No.: 13/999,635

(22) Filed: Mar. 14, 2014

Related U.S. Application Data
(60) Provisional application No. 61/852,036, filed on Mar. 15, 2013.

(51) Int. Cl. A61K 31/426; A61K 31/221; A61K 31/216

(52) U.S. Cl. CPC A61K 31/426; A61K 31/216; A61K 31/221
USPC 514/369

(57) ABSTRACT

DPN is an intractable and tormenting neurovascular and dermal disorder.

Two patients of DPN of stocking-glove pattern were treated with SAG for treatment of microvascuar coagulopathy, induction of vasodilation and possible induction of angioneogenesis; with ALC for ATP generation through mitochondrial beta-oxidation of fats; and with ALA for antioxidative stress to prevent precipitation of vascular damage and to prevent DPN recurrence. SAG+ALC was to cure maceration.

Thus, SAG+ALC+ALA was effective in mitigating and curing their distresses. It shall be one of the logical and practical means of prompt DPN therapy not only to alleviate DPN but also to lead to “cure.”

### FIELD OF THE INVENTION

Diabetic peripheral neuropathy incurs intractable pain in the limbs in stocking-glove pattern, and generates maceration and gangrene of them. It is multifaceted, and appropriate application of sarpogrelate, acetyl-L-carnitine, and α-lipoic acid halts the distress, and cures maceration before correction of hyperglycemia is attained. Recurrence of diabetic peripheral neuropathy is also preventable.

### BACKGROUND OF THE INVENTION

Recently, the number of sufferers of diabetic peripheral neuropathy (hereafter designated as DPN) has globally increased in accordance with the increase of number of patients with diabetes mellitus (hereafter designated as DM). The DPN morbidity torments patients with pain and cold sensation typically in the stocking-glove pattern.

DPN is insidious in onset and progression, and yet is tragic as it is likely to eventually lead to maceration, gangrene, and amputation of the succumbed limbs. The malady, therefore, demands therapies aiming at an acute and prompt rescue of DPN, of its cure, prevention of its recurrence, and induction of neuronal regeneration.

We propose here one such medicinal way of treatment with sarpogrelate (hereafter designated as SAG), acetyl-L-carnitine (hereafter designated as ALC), and α-lipoic acid (hereafter designated as ALA) in combination.

The etiology of DPN includes changes in the neuronal microcirculation and arterioles, neuronal loss, and attempted regeneration (Anthony et al. 2010). Prolonged hyperglycemia (glucotoxicity) and hyperinsulinemia (Sugimoto et al. 2003) are primary causes of DPN.

Therapeutically, each 200 mg dose of SAG halts DPN's pain and cold intolerance but for 3-4 hours only, although the skin turns warmer. Then, SAG not only counteracts with platelets in the neuronal arterioles and microcirculation but also dilates these vessels (Miyazaki et al. 2007). SAG monotherapy thus does not “cure” but potentially mitigates the distress in fluctuation.

Encountering the possible therapeutic effect of ALC on DPN (Sima et al. 2005), we supplemented SAG with ALC. SAG+ALC bitherapy acutely halted the distress for longer periods and cured the maceration. When withdrawal of SAG+ALC was prolonged, symptoms of the DPN relapsed.

We looked for the third agent, and ALA (Ziegler et al. 2006) was incorporated: SAG+ALC+ALA tritherapy. This appears capable of causing “cure.” ALA increases the nerve blood flow (Nagamatsu et al. 1995). Both SAG and ALC could have been synergistic in the neuronal and arterial regeneration (Taglialatela et al. 1991; Bir et al. 2008).

Anti-oxidant effects of SAG (Cao et al. 2011) and ALA (Nagamatsu et al. 1995) shall contribute to suppression of DPN relapses.

Thus, the tritherapy of DPN with SAG+ALC+ALA may be integrated in the treatment of intractable DPN.

### BRIEF DESCRIPTION OF DRAWINGS(PHOTOGRAPHS

The patent or application file contains at least one drawing executed in color. Copies of this patent or patent application publication with color drawing(s) will be provided by the Office upon request and payment of the necessary fee.

An elderly male patient of DPN with advanced DM of hyperglycemia, hypoinsulinemia, and Hgb_{A1C} elevated to 7.5%, presented himself with a typical stocking-glove pattern of DPN accompanied by maceration in the left big toe. A bitherapy of SAG+ALC halted his DPN.

FIG. 1A shows a photograph of the hand of a patient of DPN at 3 weeks of the treatment of 1500 mg ALC and 600 mg SAG. The patient declined to be photographed at the start of the treatment.

FIG. 1B shows a photograph of the hand of the same patient at 7 weeks of the ALC-SAG treatment. The skin and balls turned to normal skin color.

FIG. 1C shows a photograph of the hand of the same patient at 2 weeks of epalrestat (hereafter designated as EPR) and ALC 7 weeks after the ALC-SAG.

FIG. 1D shows a photograph of the foot of the same patient of DPN at 3 weeks of ALC-SAG. The first toe is deeply reddened. It is tender because of maceration in the depth from an advanced DPN.

FIG. 1E shows a photograph of the foot at 7 weeks of the same ALC-SAG therapy. The redness of the toe turned to normal skin color. The skin was non-tender as the maceration dissolved.

FIG. 1F shows a photograph of the same foot at 2 weeks of ALC-EPR therapy after the 7 weeks of ALC-SAG. EPR appeared synergistic with ALC in the treatment of DPN.

FIGS. 2A-2D show another case of the DPN of stocking-glove pattern in a 54-year-old male pre-diabetic athlete. He was hyperinsulinemic on an OGT along with hyperglycemia. His Hgb_{A1C} was normal at 5.7%. Neuronal conduction velocicty reduced was reduced to the bottom normal, and his diagnosis was DPN.

FIG. 2A shows hands were discolored purplish-red and swollen.

FIG. 2B shows feet also were discolored and swollen. His diagnosis of DPN was made based on bottom-normal nerve conduction veleocity of 40.4 m/sec as well as hyperinsulinemia on OGT. Therapy of SAG+ALC resolved the appearance and improved the sensation of pricking pain and tightness.

FIGS. 2C and 2D show hands and feet after 3 years and half with 300 mg SAG+1500 mg ALC plus 4 months with 300 mg SAG+1500 mg ALC+600 mg ALA for 4 months. The skin was of normal skin color without swelling.

### DETAILED DESCRIPTION OF A PREFERRED EMBODIMENT

We were keenly interested in SAG's acute but passing rescue of DPN distress in a patient of DPN of stocking-glove pattern. This was the beginning of the breakthrough. Our second surprise was its prolonged rescue achievable with added ALC. The third of the breakthroughs was ALA's prevention of DPN recurrence and innocent maintenance to “cure.” That SAG+ALC+ALA principle shall represent a prototype practice of treating DPN.

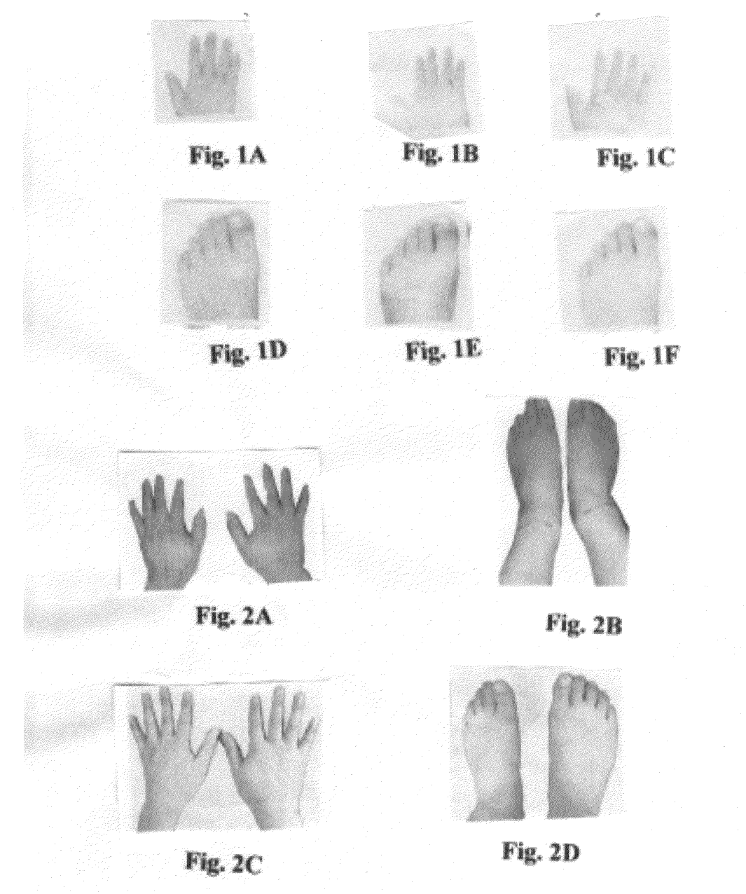

The present DPN-relieving invention includes mainly four parts: (1) rescue with SAG-ALC of the tormenting DPN distress of pain, cold sensation, and maceration in the stocking-glove pattern; (2) expansion of the SAG-ALC regimen to include ALA; (3) extended use of the SAG+ALC+ALA principle for prevention of recurrence and maintenance; and (4) an extended use of the principle for induction of neuronal regeneration with SAG+ALC+ALA.
(1) Prompt halt of DPN symptoms with the application of SAG in order to amend the pre-neuronal coagulative microangiopathy and with ALC to amend the neuronal mitochondrial fatty acid transport across the carnithine window so as to help generating ATP through β-oxidation of fatty acids: the SAG+ALC principle.
   A 70-year-old man with a 10-year history of DM developed signs and symptoms of DPN of the stocking-glove pattern and maceration in the big toe in the depth. His hyperglycemic control had been inadequate, but he did not have diabetic nephropathy or retinopathy as regularly checked.
   He was first given SAG at 600 mg per day. The dosage could be 100 to 200 mg three times a day unless contraindicated by hemorrhagic diathesis. He was relieved of the distress but limitedly for 2-3 hours of post-dose. The patient was greatly depressed to learn that neuronal loss that could have already taken place might not be recovered with the SAG (Srinivasan et al. 2000)).
   ALC was added at 1500 mg per day. The dosage range could be 500 to 4500 mg per day in the hope of eventual induction of neuronal regeneration (Tagliatela et al. 1991; Ishii et al. 2000). ALC at 1500 mg per day added to 600 mg SAG resulted in a prompt and marked symptomatic improvement (FIGS. 1a and 1d). Thus, the SAG and ALC appeared to work synergistically. Within 2 months of this combination treatment, the soft swelling due to maceration in the depth of the big toe resolved (FIG. 1e). This ALC-SAG combination therapy may be continued as long as one's DPN status persists. His DPN halt was attained before a satisfactory control of euglycemia, and normal Hgb_{A1c} was achieved.
   He was then placed on EPR at 100 mg three times a day just for a purpose of trial, in addition to ALC after 7 weeks of the SAG+ALC. Even further synergistic benefits were observed as his neurological symptoms continued to improve. The EPR dosage could be from 150 to 600 mg per day. In our experience, SAG+ALC+EPR combination will also be beneficial, although we do not have any photographs of follow-up to show.
   This lower overall dosage was associated with continued improvement and was well tolerated. The skin temperature at the lower shanks was almost normal at 35.6 degrees Celsius (Ogawa et al. 1999).
   Summing up, the therapy of our SAG+ALC regimen targeting the pre-neuronal coagulative microangiopathy and neuronal dendritic mitochondrial dysfunction halted his DPN symptoms in the stocking-glove pattern in a 70-year-old diabetic whose diabetic control had not been satisfactory enough: the left hand (FIGS. 1a, 1b, and 1c) and left foot (FIGS. 1e, 1e, and 1f). The tender maceration deep in the big toe was cured.

We realize that single SAG for 3 months appreciably but passingly alleviated his DPN symptoms of pain, cold sensation, and numbness. Nonetheless, our SAG+ALC combination therapy promptly halted these symptoms.

In this way, the combined SAG+ALC charge was concluded to effectively rescue DPN of stocking-glove pattern accompanied by maceration through amendment to the pre-neuronal coagulative microangiopathy (SAG) and neuronal mitochondriopathy (ALC). That principle was maintained until he developed hypocholesterolemia of 80 mg per dl 3 months later.

Neurological recurrence occurred several months after the cessation of the SAG+ALC. A second course of the SAG+ALC were effective, but did not prevent recurrence of the DPN in due course of time. Then, α-lipoic acid (thereafter designated as ALA) at 600 mg per day was given along with SAG. This combination also promptly halted his DPN symptoms. Being encouraged by that result, a third principle of SAG+ALC+ALA was tried on him for 6 months. That principle proficiently prevented any signs and symptoms of his DPN recurrence for 5 years as followed up. Had he been cured of the disorder? His total cholesterol levels continued to be within normal range thereafter in spite of ALC maintained.

Clinically, we had better watch out for signs and symptoms of hemorrhagic diathesis with a chronic use of SAG, and for development of hypocholesterolemia in the case of ALC.

FIGS. 2A and 2B show hands and feet of DPN of stocking-glove pattern resulting from hyperinsulinemia in a 54-year-old athlete. He used to overeat as an athlete in his youth. He is pre-diabetic but of elevated insulin response to an OGT. He was treated with SAG at 300 mg per day and 1500 mg ALC. The dermal discoloration and swelling resolved shortly (FIGS. 2C and 2D). However, pricking pain and tightness in the limbs did not dissolve with 300 mg SAG+1500 mg ALC+600 mg ALA for 1 year. We suspected his SAG dosage was inadequate, and his SAG dosage is being double to 600 mg per day in order to stimulate neoangiogenesis.

### Biological Basis of Pre-Neuronal Diabetic Coagulative Micro-Angiopathy

DM induces platelet aggregation (Sagel et al. 1975). SAG inhibits that morbidity. It dilates arterioles and increases local blood circulation (Ogawa et al. 1999; Miyazaki et al. 2007; Bir et al. 2008), raising the skin temperature. However, in most advanced DPN patients, their DPN symptoms are not satisfactorily rescued with single SAG usage. Single ALC is effective in the treatment of DPN (Sima et al. 2005; De Grandis and Minardi 2002), but even with high dosages, it took too long a time before the treated patients admitted they were relieved at all. ALA monotherapy is also reported to alleviate DPN relatively shortly (Ziegler et al. 2006). We claim that SAG might be synergistic with ALC+ALA, and intensify and accelerate their potentials. ALA dendritic is to counteract oxidative stress, and mitigates atherosclerotic changes (Ziegler et al. 2004) so that neuronal and dendritric microcirculation shall also be improved.

Our SAG-ALC combination promptly halted and was rescuing. The SAG-ALA was also promptly rescuing. Both ALC and ALA encourage β-oxidation of fatty acids in the mitochondria. With those advanced DM patients, serum ALC has been reported to decrease (Poorabbas et al. 2007). Therefore, single ALC supplementation is expected to encourage β-oxidation of fatty acids as ALC, a cotransporter of fatty acids helping them to travel across the mitochondrial inner membrane (carnitine window) (Botham and Mayes 2006). Effectiveness of these agents will be greatly reinforced when they are combined with SAG, a pre-neuronal means to increase drug delivery to the DPN neurons, and rescue the DPN distress.

Coagulative microangiopathy may effectively be treated with these agents that will prevent platelet aggregation and dissolve the coagula, beginning with heparin and aspirins (Table 1). However, diabetic angiopathy includes vascular sclerosis to any appreciable degree. In order to exploit the maximum therapeutic results, we have to employ arterial dilators as well. Vascular dilation will itself help alleviating cold sensation along with increasing drug delivery.

Beside SAG, ethyl icosapentate, argatroban hydrate, and cilostazol are known to exert vascular dilating action along with anti-coagulopathy (Table 1), and they may deserve clinical evaluation in combination with ALC and/or ALA.

SAG is different from those agents. It potentially increase neoangiogenesis (Bir et al. 2005), so that dendritic microcirculation shall be reconstructed. Our use of SAG inpatients of DPN shall help their blood sugar control through persistent insulin-sensitizing effect (Kokubu et al. 2006). Thus, SAG+ALC+ALA shall exert a multifaceted beneficial action on DPN changes.

TABLE 1
Anti-coagulatives that have vasodilating activity.

| | Anti-platelet | Arteriolar | Neo- | Insulin- |
|---|---|---|---|---|
| | Aggregation | dilation | angiogenesis | sensitization |
| Heparin | Yes | No | No | No |
| Bufferin | Yes | No | No | No |
| Epadel | Yes | Yes | No | No |
| (ethyl | | | | |
| icosapentate) | | | | |
| Novastan | Yes | Yes | No | No |
| (argatroban | | | | |
| hydrate) | | | | |
| Anplag | Yes | Yes | Yes | Yes |
| (sarpogrelate) | | | | |
| Pletaal | Yes | Yes | No | No |
| (cilostazol) | | | | |

### Biological Basis of Neuronal Diabetic Neuropathy

It is known that hyperinsulinemia may become chronic with high protein diets (Carr et al. 2008), which can give rise to “DPN” (Sugimoto et al. 2003, 2008; Kim et al. 2011) even prior to the emergence of clinical diabetes. This may have been the case with our first patient. The second, more elderly patient, developed full DM along with β-cell exhaustion, and must have experienced periods of hyperinsulinemia that precipitated damage to the peripheral nerves for this DPN to emerge later.

Our present experience with the one pre-diabetic hyperinsulinemic, and the other hypoinsulinemic diabetes, led us to consider that the latter must have exhausted the pancreatic β-cells through hyperinsulinemia during his pre-diabetic period. Therefore, the occurrence of “pre-diabetic” hyperinsulinemia should be considered in clinical settings since it may precipitate DPN.

Starting with a rather conventional SAG monotherapy, we expanded the regimen to construct a potent combination therapy for relief of painful DPN and maceration, which resulted in apparent cure lasting at least for 5 years on follow-up. Effective combination therapies, based on our present cases, may comprise SAG+ALC, and SAG+ALC+ALA, and possibly SAG+ALC+EPR, and SAG+ALC+EPR+ALA. Further, it is noteworthy that control of hyperglycemia was not a prerequisite for the resolution of symptoms in neither case.

The etiology of the DPN includes changes in the neuronal arterioles, neuronal loss, and attempted regeneration, presumably from prolonged glucotoxicity (Anthony et al. 2010). The pathophysiology of DPN progression is characterized by hypertriglycemia in the presence of inadequate hyperglycemic control (Wiggin et al. 2009). In cases of DM, ALC is also deficient (Tamamogullari et al. 1999; Poorabbas et al. 2007) such that ALC's control of dyslipidemia would also help improving DPN (Malone et al. 1996; De Grandis and Minardi 2002).

However, Wiggin et al. could not produce the expected effect of ALC monotherapy on DPN progression (2009), in spite of its acclaimed efficacy in transporting fatty acid across the mitochondrial inner membrane for β-oxidation and ATP production (Botham and Mayes 2006). ALA also helps improving neurovascular function in DM rats (Cameron et al. 1998).

Increased oxidative stress in DM precipitates neuronal apoptosis (Russell et al. 1999; Srinivasant et al. 2000; Vincent wet al. 2002), and initiates arteriolosclerosis. Persistent hypertriglycemia may result in pathologically appreciable changes, such as coagulative microangiopathy, i.e., neuronal arteriolosclerosis (Chitre et al. 1988; Sagel et al. 1975; Ferroni et al. 2004; Sudic et al. 2006; Anthony et al. 2010). An experimental cotherapy of ALC and ALA in old rats is capable of reducing oxidative stress (Hagen et al. 2002).

When our patient's DPN recurred with cold intolerance, he was then treated with the tritherapy of SAG+ALC+ALA, which resolved all symptoms and appeared to provide a cure lasting at least for 5 years with the maintenance use of the tritherapy.

The mechanisms behind the efficacy of the present tritherapy may include the following:
(1) The circulatory insufficiency from coagulative microangiopathy may be addressed by SAG which inhibits platelet aggregation, dilates the vessels (Hara et al. 1991), and activates endothelial regeneration (Bir et al. 2008). Dilation of the neuronal arterioles may be induced by ALC, which support the generation of ATP by enhancing fatty acid passage across the mitochondrial inner membrane for β-oxidation (Botham and Mayes 2006).
(2) Obstructive vascular pathology may be precipitated through oxidative stress (Ziegler et al. 2004) and hypertriglycemia (Wiggin et al. 2009), and both ALC and ALA will be protective against those insults (Nagamatsu et al. 1995; Botham and Mayes 2005; Cao et al. 2011).
(3) The neuronal loss through apoptosis in DM could also be reduced by both ALC (Tagliatela et al. 1991; Galli and Fraterlli 1993; Ishii et al. 2000; Forloni et al. 2001) and ALA (Ziegler et al. 2004).
(4) A long-term cure of DPN may be realized through neuronal regeneration induced by ALC (Tagliatela et al. 1995; Sima et al. 2005; Bir et al. 2008). The cells in regeneration are highly sensitive to oxidative stress and both ALC and ALA are protective when they are given for maintenance (Ziegler et al. 2004; Cao et al. 2011).

Thus, these globally interactive principles contained in the present SAG+ALC+ALA tritherapy are potentially curative for the multifaceted disease of DPN. Further, this therapy may be promising in selected cases of ASO (arteriolsclerosis obliterans), and possibly Alzheimer's disease (Chan et al. 2008).

We suspect that the development of the stocking-glove pattern seen with DPN is an evolutionary legacy of quadrupedalism: the peripheral nerves of the limbs are insulin-insensitive (Bender and Mayes 2006) so as to better adapt to glucose demand for fast running while chasing or fleeing. Resultantly, hyperinsulinemic insults will be more demanding to the nerves of the limbs in the development of DPN (Sugimoto et al. 2003, 2008; Kim et al 2011). It is noteworthy that the son of our more elderly patient was found to be hyperinsulinemic without any signs of the possibility of developing DPN based on the present rationale. SAG is capable of inducing persistent insulin sensitization (Kokubu et al. 2006), supporting our maintenance use of SAG.

We are keenly interested in how the hyperinsulinemia could have triggered the early pre-daibetic development of DPN. We may discuss the matter in two different categories: (I) insulin and insulin receptors, and (II) neuronal, i.e., anatomy and physiology.

I. Dobretsov et al. Discuss the Insulin-Side (2007):
1) Insulin circulates in the peripheral nervous system (thereafter designated as PNS) as it does in the general circulation;
2) As it reaches the PNS, it binds to the insulin receptors (thereafter designated as IR-A) on the endothelial cells, paranodal loops of Schwann cells, and the primary sensory nerves of medium and small size: the target sites of the stocking-glove pattern of DPN;
3) IR-A is densely distributed in the PNS;
4) IR-A's affinity to insulin is almost identical with IR-B in the liver, skeletal muscle, and fat cells. Then, such insulin abundance in the treatment of type 1 DM and in the pre-daibetic hyperinsulinemia of type 2 DM candidates may trigger the development of DPN;
5) To the contrary, the liver, skeletal muscle and fat cells are not daunted at the hyperinsulinemia of insulin resistance in a practical sense;
6) SAG shall counteract the damaging hyperinsulinemia by inducing persistent insulin-sensitization (Kokubu et al. 2006).

However, their illustrative description may not be appropriate enough to fully explain the specific DPN in the PNS.

### II. Neuron-Side Story:

The observed DPN halt was prompted in both SAT+ALC and SAG*ALA. The SAG monotherapy induces insulin-sensitization, and the effect is maintained (Kokubu et al 2006). An angiogenetic effect of SAG (Bir et al. 2008) will contribute to the recovery of local circulation. An increase of ATP generation is probable with the ALC that helps fatty acid transport across the inner membrane of the mitochondria, and thus permits the β-oxidation to generate ATP in the presence of hyperlipidemia of DM.

Owing to the increase of oxidative stress in DM, hyperinsulinemia and oxidative burden will incur damage to the mitochondria in the neuronal dendrites. As the dendritic mitochondria are numerous (Allman 2000), ALC's therapeutic recovering effect, therefore, can be prompt. We may also expect that ALC's activation of neuronal regeneration (Tagliatela et al. 1991, 1995), thus contributing to the cure of DPN.

ALA is a potent biological anti-oxidant against DM's oxidative stress, and its prompt relief of the DPN together with SAG may be perpetuated by its additional property of angiogenesis (Shakher and Stevens 2011; Gobidi et al. 2011).

Once DPN has manifested, it will be aggravated by hyperglycemia, and especially hypertriglycemia (Wiggin et al. 2009). It should also be stressed that DPN and ASO are much more likely to arise among pre-diabetic hyperinsulinemia.

The multifaceted nature of DPN requires an equally multifaceted therapy, and it was this realization that guided our exploration of the present therapies. It is our hope and expectation that this novel approach will also be useful in the prevention and therapy of ASO.

### The Potential Neuronal Regeneration:

DPN or neuronal degeneration is one of the serious complications of DM (Yagihashi 1995), and amputation of limbs for diabetic arteriosclerosis obliterans (ASO) is a common tragic outcome. Unfortunately, regeneration of neurons may not occur with either single treatment with EPR or SAG.

The potential benefits of ALC for DPN have long been discussed (Nagamatsu et al. 1995). It is thought to provide the dendritic mitochondria (Allman 2000) with surplus ALC, and may encourage appreciable neuronal regeneration over a prolonged period of time (Chitre et al. 1988; Botham and Masyes 2006; Chan et al. 2008). Further, SAG may prepare damaged neurons to take advantage of improved circulation. Thus, we may expect a greater synergistic effect between ALC and SAG, and it was the case with our elderly patient: his DPN distresses were thoroughly dissolved in the due course of time.

### REFERENCES

1. Allman J M (2000) Evolving Brains. Scientific American Library, Freeman, N.Y.
2. Anthony D C, Frosch M P & Girolami U (2010) Peripheral Nerve and Skeletal Muscle. In: Robbins and Cotran: Pathologic Basis of Disease, 8^{th} ed.; Kumar V, Abbas A K, Fausto M & Aster J C, eds. Saunders-Elsevier, Philadelphia, 2010. pp 1257-1277.
3. Bender D A & Mayes P A (2006) The pentose phosphate pathway and other pathways of hexose metabolism. In: Harper's Illustrated Biochemistry, 27^{th} ed.; Murray R M, Graner D K & Rodwell V W eds. Lange/McGraw-Hill, New York. Pp 177-186.
4. Bir S C, Fujita M, Marui A, Hirose K, Arai Y, Sakaguchi H, Huaang Y, Esaki J, Ikeda T, Tabata Y and Komeda M (2008) New therapeutic approach for impaired arteriogenesis in diabetic mouse hind limb ischemia. Circ J 2008; 72: 633-640.
5. Botham K M & Mayes P A (2006) Oxidation of fatty acids: Ketogenesis. In: Harper's Illustrated Biochemistry, 27^{th} ed.; Murray R K, Graner D K & Rodwell V W eds. Lange?McGraw-Hill, New York. pp 187-195.
6. Cameron N F, Cotter M, Horrobin D H & Tritscher H T (1998) Effects of α-lipoic acid on neurovascular function indiabetic rats: Interaction wioth essential fatty acids. Diabetollogia 41: 390-399.
7. Cao Y, Qu H J, Li P, Wang C B, Wang L X & Han Z W (2011) Single dose administration of L-carnitine improves antioxidant activities in healthy subjects. Tohoku J Exp Med 224: 209-213.
8. Carr R S, Larsen M O, Winzell M S, Jelic K, Lindgren D, Deacon C F & Ahren B (2008) Incretin and islet hormonal responses to fat and protein ingestion in healthy men. Am J Physiol Endocrinol Metab 295: E779-E784.
9. Chan A, Paskavitz J, Remington R, Rasmussen S & Shea T B (2008) Efficacy of a vitamin/nutriceutical formulation for early Alzheimer's disease: a 1-year-open-label pilot study with a 16-month caregiver extension. Am J Alzheimer's Dis Other Demen 23: 571-585.
10. Chitre A P & Veloskar D S (1988) Role of platelets in diabetic microangiopathy—an additional factor. Angiology 39: 458-465.
11. De Grandis D & Minardi C (2002) Acetyl-L-carnitine (levacecarnitine) in the treatment of diabetic neuropathy: A long-term, randomized, double-blinded, placebo controlled study. Drugs R & D 3: 223-231.
12. Dobretsov M, Romanovsky D & Stimers J R (2007) Early diabetic neuropathy: Triggers and mechanisms. World J Gastroeenterol 13: 175-191.
13. Ferroni P, Basilli S, Fale A & Davi G (2004) Platelet activation in type 2 diabetes mellitus. 2 Thromb Haemost: 2: 1282-1291.
14. Forloni P, Angeretti N & Smiroldo S (2001) Neuroprotective sctivity of acetyl-L-carnitine: studies in vitro. J Neurosci Res 37: 92-96.
15. Galli G & Fratelli M (1993) Activation of apoptosis by serum deprivation in teratocarcinoma cell line: inhibition by L-carnitine. Exp Cell Res 204: 54-60.
16. Gobidi S, Badram M & Laher I (2011) Diabetes and alpha lipoic acid. Front Pharmacol 2: 69. Epub 2011 Nov. 7.
17. Hagen T M, Lu J, Lykkasfeldt J, Wehr C M, Ingersoll N T, Vinarsky V, Barthromew J C & Ames B N (2002) Feeding acetyl-L0carnitine and lipoic acid to old rats significantly improve metabolic function with decreasing oxidative stress. Proc Natl Acad Sci USA 99: 1870-1875.
18. Hara H, Kitajima A, Shimada H & Tamao Y (1991) Antithrombotic effect of MCI-9042, a new antiplatelet agent on experimental thrombosis models. Thromb Haemost (Stuttgart) 65: 484-488.
19. Ishii T, Shimpo Y, Matsuoka Y & Kinoshita K (2000) Anti-apoptotic effect of acetyl-1-carnitine and l-carnitine in primary cultured neurons. Jpn J Pharmacol 83: 119-124.
20. Kim B, McLean L L, Philip S S & Feldman E L (2011) Hyperinsulinemia induces insulin resistance in dorsal root ganglion nerves. Endocrinology 152: 3638-3647.
21. Kokubu N, Tsuchihashi K, Yuda S, Hase M, Eguchi M, Wakabayashi T, Hashimoto A, Nagao K, Tsuzuki M, Wakabayashi Ch & Shimamoto K (2006) Persistent insulin-sensitizing effects of sarpogrelate hydrochloride, a serotonin 2A receptor antagonist, in patients with peripheral arterial disease. Circ J 70: 1451-1456.
22. Malone J I, Lowitt S, Salem A F, Miranda C, Korthals J K & Carver J (1996) The effect of acetyl-L-carnitine and sorbitol on peripheral nerve structure chemistry, and function in experimental diabetes. Metabolism 45: 158-162.
23. Miyazaki M, Higashi Y, Goto C, Chayama K, Yoshizumi M, Sanada M, Orihashi K & Sueda T (2007) Sarpogrelate hydrochloride, a selective 5-HT2A antagonist, improves vascular function in patients with peripheral arterial disease. J Cardiovasc Pharmacol 2007; 49: 221-227.
24. Nagamatsu M, Nickander K K, Schmelzer J D, Raya A, Wittrock D A, Tritschler H & Low P A (1995) Lipoic acid improves distal nerve conduction in experimental diabetic neuropathy. Diabetes Care 18: 1160-1167.
25. Ogawa S, Takeuchi K, Sugimura K, sato C, Fukuda M, Lee R, Ito S & Sato T. The 5-HT2 receptor antagonist sarpogrelate reduces urinary and plasma levels of thromboxane A₂ and urinary albumin excretion in non-insulin-dependent diabetes mellitus patients. Clin Exp Pharmacol Physiol 26: 461-4, 1999.
26. Okuyama S, Okuyama J, Lu J, Lu H & Ichiki M (2014) Sarpogrelate, acetyl-L-carnitine, and α-lipoic acid in combination promptly halt diabetic peripheral neuropathy, and may lead to its cure. Tohoku J Exp Med, to be submitted.
27. Poorabbas A, Fallah D, Bagdadchi J, Jabbari R, Aliasgarzadeh A, Asadi Y, Koushavar H & Vahed Jabbari M. (2007) Determination of free L-carnitine levels in type II diabetic women with and without complications. Eur J Clin Nutr 61: 892-5, 2007.
28. Russell J W, Sullivan K A, Windebank A J, Herrmann D N & Feldman E L (1999) Neurons undergo apoptosis in animal and cell culture models of diabetes. Neurobiol Dis. 6: 347-363.
29. Sagel J, Colwell J A, Crook L & Laimins M (1975) Increased platelet aggregation in early diabetes mellitus. Ann Int Med 82: 733-738.
30. Shakher J & Stevens M J (2011) Update on trhe management of diabetic poluneuropathy. Diabetes Metab Syndr Obes 4: 289-305.
31. Sima A A F, Calvani M, Mehra M & Amato A. (2005) Acetyl-L-Carnitine Study Group. Acetyl-L-carnitine improves pain, nerve regeneration and vibratory perception in patients with chronic diabetic neuropathy. Diabetes Care 28: 89-94.
32. Srinivasan S, Stevens M & Wiley J W. (2000) Diabetic peripheral neuropathy: evidence for apoptosis and associated mitochondrial dysfunction. Diabetes 49: 1932-1938.
33. Sudic D, Razmara M, Forslun M, Ji Q, Hjemdahl P & Li N (2006) High glucose levels enhance platelet activation: involvement of multiple mechanisms. Br J Haematol 133: 315-324.
34. Sugimoto K, Baba M, Suda T, Yasujima M & Yagihashi S (2003) Peripheral neuropathy and microangiopathy in rats with insulinoma: association with chronic hyperinsulinemia. Diabet Metab Res Rev 19: 392-400.
35. Sugimoto K, Rashid Ib, Shoji M, Suda T & Yasujima M. (2008) Early changes in insulin signaling and pain sensation in streptozocin-induced diabetic neuropathy in rats. J. Pain 9: 237-45.
36. Taglialatela G, Angelucci L, Ramacci N T, Werbach-Perez K, Jackson G R & Perez-Polo J R. (1991) Acetyl-L-carnitine enhances the response of PC12 cells to nerve growth factor. Dev Brain Res 59: 221-30.
37. Taglialatela G, Navarra D, Olivi A, Ramacci M R, Werbach-Perez K, Perez-Polo J R E. Angelucci L (1995) Neurite outgrowth in PC12 cells stimulated by acetyl-L-carnitine arginine amide. Neurochem Res 20: 1-9.
38. Tamamogullari N, Silig Y, Icagstoglu S & Atalay A (1999) Carnitine deficiency in diabetes mellitus complications. J Diabet Complicat 13:51-253.
39. Vincent A M, Brownlee M & Russell J W (2002) Oxidative stress and programed cell death in diabetic neuropathy. Ann N Y Acad Sci 959: 368-383.
40. Wiggin T D, Sullivan K A, Pop-Busui R, Amato A, Sima A A F & Feldman E L (2009) Elevated triglycerides correlate with progrerssion of diabetic neuropathy. Diabetes 58: 1534-1640.
41. Yagihashi S. (1995) Pathology and pathogenetic mechanisms of diabetic neuropathy. Diab Metab Rev 11: 193-225, 1995.
42. Ziegler D, Sohr C G H & Nourooz-Zadeh J (2004) Oxidativestress and antioxidant defense in relation to the severity of diabetic neuropathy and cardiovascular autonomic neuropathy. Diabetes Care 27: 2178-2183.
43. Ziegler D, Ametov A, Barinov A, Dyck P, Grieva I, Low P A, Munzel N, Raz I, Novosadova M, Maus J & Samugullin R (2006) Oral treatment with alpha-lipoic acid improves symptomatic diabetic neuropathy. Diabetes Care 29: 2365-2370.

1. A method of treating diabetic neuropathy, including a first stage of treatment with sarpogrelate (SAG) for a period of time, a second stage of treatment with SAG and acetyl-L-carnitine (ALC) for a period of time; and a third stage of treatment with ALC and epalrestat (EPR) for a period of time.

2. The method as claim 1, in which in the first stage, the dosage of SAG is 600 mg per day.

3. A method as claim 1, in which, in the second stage of treatment with ALC, the ALC dosage was 1500 mg per day.

4. A method as claim 1, in which, in the third stage of treatment with EPR, the dosage was 300 mg per day.

5. A method as claim 1, in which in the fourth stage of treatment with ALA, the dosage was 600 mg per day.

6. A photographic evidence as claim 1, in which the changing treatment of DPN with SAG+ALC in a case of hyperinsulinemic pre-diabetic DPN.

7. A photographic evidence as claim 1, in which the charging treatment of DPN with ALC and SAG was shown to promptly heal dermatological abnormalities of skin discoloration and maceration in the depth along with rescuing neurological distresses.

8. A method as claim 1, in which any combinations of SAG with the metabolic modifiers as ALC, or EPR, especially the SAG-ALC appeared promising in the treatment of DPN with eventual attainment of neuronal regeneration.

9. A method as claim 1, in which the contribution of SAG+ALC+ALA halted the DPN, and seemed to have cured it for the 5 years of follow-up without signs and symptoms of recurrence: eventual attainment of neuronal regeneration with this SAG+ALCX+ALA principle.

10. This SAG+ALC+ALA principle may be applicable to prevention of untoward development of DPN candidates as cases of hyperinsulinemic insulin resistance, those of pre-diabetic chronic post-prandial hyperinsulinemia, those of ASO, and dementias of AD and others.

11. The method as claim 1, in which in the first stage, the dosage of SAG is 300 mg per day.
